(19) United States
(12) Patent Application Publication
(10) Pub. No.: US 2010/0112579 A1
(43) Pub. Date: May 6, 2010

(54) METHOD AND DEVICE FOR THE DETECTION OF GENETIC MATERIAL BY POLYMERASE CHAIN REACTION

(75) Inventors: Dolores Verdoy Berastegui, Zamudio (Vizcaya) (ES); Garbine Olabarria De Pablo, Zamudio (Vizoaya) (ES); Jesus Miguel Ruano Lopez, Arrasate-Mondragon (Guipuzcoa) (ES); Javier Berganzo Ruiz, Arrasate-Mondragon (Guipuzcoa) (ES)

Correspondence Address: FULWIDER PATTON LLP, HOWARD HUGHES CENTER, 6060 CENTER DRIVE, TENTH FLOOR, LOS ANGELES, CA, 90045, US

(73) Assignees: FUNDACION GAIKER, Zamudio (Vizcaya) (ES); SOCIEDAD COOPERATIVA IKERLAN, Arrasate-Mondragon (Guipuzcoa) (ES)

(21) Appl. No.: 12/593,283

(22) PCT Filed: Mar. 26, 2007
(86) PCT No.: PCT/ES07/00163

§ 371 Date: Dec. 4, 2009

(51) Int. Cl. C12Q 1/68; C12M 1/38

(52) U.S. Cl. 435/6; 435/286.1

(57) ABSTRACT

The present invention relates to a method and a portable device to detect a genetic material in a biological sample using the technique known as PCR (Polymerase Chain Reaction). The device comprises a reaction chamber which incorporates means of heating arranged to heat said chamber. The detection method is characterized in that the main phases are performed in the reaction chamber. The miniaturized system has the object of increasing efficiency, simplicity of use and the portability of the PCR in comparison with laboratory scale analysis.

### OBJECT OF THE INVENTION

The present invention relates to a method and to a portable device or microdevice to specifically detect genetic material in a biological sample using the technique known as PCR (Polymerase Chain Reaction).

The microdevice has the object of increasing the efficiency, simplicity of use and the portability of the PCR in comparison with analysis on a laboratory scale. The microdevice makes it possible to quickly diagnose the presence of a determined sequence of oligonucleotides (DNA and RNA), by the technique of real-time PCR in a final volume, for example, of 10 microlitres and in less than 30 minutes.

### BACKGROUND OF THE INVENTION

The technique known as PCR (Polymerase Chain Reaction) reproduces the natural DNA replication system for a determined fragment of the genome, making it possible to produce many copies of a determined DNA sequence, provided that the target sequence one wants to amplify is found in the biological sample. These methods comprise, therefore, a first phase of concentration in the sample of the fraction containing the specific sequence to identify, followed by a second phase, if necessary, of rupture or lysis to permit the accessibility of said sequence to finally be subjected to PCR which permits its specific identification. The PCR treatment requires heating phases. The detection phase of the amplified product is generally performed by optical means.

The detection of genetic material is, therefore, based on its amplification, since in the initial sample it is found in very small quantities which cannot be detected, whilst, through PCR reaction, the genetic material starts its amplification until it can be detected (if it is not present in the sample, obviously the detection is not made).

Typically, the PCR reaction in a microdevice is performed in a micro-chamber (within a chip or plastic support) which has a small inlet for the sample and a second orifice for the outlet thereof. Generally, the devices used are very complex since they comprise several chambers whereby the sample is made to pass to perform the concentration of the target analyte, PCR reaction and detection, which slows down the process. The means of heating are typically external to the chamber or plastic support wherein the PCR reaction is produced, and they do not provide a suitable and fast heating in all parts of the chamber where the reaction occurs.

Likewise, various methods are known which permit achieving a preconcentration in the biological sample of the fraction which contains the specific sequence for the PCR reaction. In this sense, superparamagnetic particles are also known, coated by a specific antibody which permit the concentration of the biological sample. These particles have a magnetic part, for which reason they can be detected or separated by a magnetic field. Magnetic particles are used, for example, in U.S. Pat. No. 881,541, US 2004/108253, U.S. Pat. No. 5,795,470, US 2005/208464 or U.S. Pat. No. 6,159,378.

Various procedures are also known of optical detection procedures, for example by fluorescence (EP 1 550 858, WO 2005/023427 or U.S. Pat. No. 6,814,934).

The means of heating are very diverse, but always external to the plastic support or chip which incorporate the chamber wherein the PCR reaction is produced.

### DESCRIPTION OF THE INVENTION

The present invention performs the concentration of the sample by superparamagnetic particles, specific identification by real-time PCR reaction and detection by fluorescence. One of the principal characteristics of the invention is that the concentration, the lysis, when necessary, the heating, the PCR reaction, and the detection by fluorescence, are performed in the same micro-chamber, i.e. without the genetic material leaving this micro-chamber.

In particular, the superparamagnetic particles are mixed with the sample to be analysed, which permits enrichment in the fraction which contains the target sequence. The sample is introduced, with the superparamagnetic particles, inside a micro-chamber. Magnets are applied on the opposing surfaces of the micro-chamber, at a very short distance, so that the magnetic field generated retains the superparamagnetic particles (whilst the rest of the sample exits the micro-chamber). The reagents are introduced in the micro-chamber which will produce the PCR reaction and the fluorescence markers, the magnets are removed, the inlet/outlet is plugged and a heating profile is then applied through a heating device situated beside the chamber, producing the amplification of the genetic material in the same micro-chamber.

Then, the device is taken to optical means which make it possible to capture the fluorescence and thus detect the presence of the genetic material sought, without this or the superparamagnetic particles leaving the micro-chamber.

Another of the aspects of the invention relates to devices wherein the detection process is carried out. The device, in addition to containing the micro-chamber wherein the PCR reaction is produced, receives the heat generated by the means of heating which are composed of a series of titanium/platinum electrodes, as well as the means necessary to perform all the phases of the detection process in the same reaction micro-chamber.

More specifically, one of the aspects of the invention relates to a device for the detection of genetic material by polymerase chain reaction, which comprises a substrate wherein a reaction chamber as well as an inlet micro-conduit and an outlet micro-conduit are formed, respectively for the inlet and outlet of a sample to be analysed from said chamber. The device incorporates means of heating suitably disposed for heat to uniformly heat said chamber.

For the introduction of the sample and of the PCR reagents in the micro-chamber, said substrate (chip or plastic support) is retained with dismountable character in an encapsulate formed by an upper base and a lower base positioning the micro-chamber between said upper and lower bases. The means of heating may be integrated in one of these bases to heat said chamber, or be integrated in the substrate itself wherein the reaction chamber is formed.

The chamber is accessible through the upper and lower bases by corresponding openings existing in said bases, with the object of performing various phases of the process on the chamber, for example the application of a magnetic field by magnets and the optical detection.

The device may have a temperature sensor to measure the temperature in the reaction chamber, as well as electric contacts to electrically supply the means of heating and provide a connection with the temperature sensor.

Said means of heating comprise a plurality of conductive wires connected between two terminals.

The device has means which make it possible to produce a uniform current distribution in said conductive wires, so that each of said wires generates a very similar quantity of heat. In this way, and due to the uniform distribution of the wires under the whole surface of the reaction chamber, a uniform heating is provided throughout the reaction chamber.

Another aspect of the invention relates to an apparatus for the detection of genetic material by a polymerase chain reaction, which incorporates the aforementioned device, and it is complemented with electronic means external to said device to control the temperature produced by the means of heating of the reaction chamber, as well as a fluorescence measuring system.

Another aspect of the invention is related to a plastic support for the specific detection of genetic material by polymerase chain reaction, which comprises an upper face and a lower face, and it is characterized in that between said upper and lower faces it incorporates a reaction chamber and an inlet micro-conduit and an outlet micro-conduit connected to said chamber. The reaction chamber and the inlet and outlet micro-conduits, are accessible from at least one of said faces to be able to perform the detection process on the chamber.

An object of the invention is also a method for the specific detection of genetic material by polymerase chain reaction, which is characterized in that the main phases of the method are carried out in the same reaction chamber.

In a more detailed way, the method comprises introducing in a reaction chamber a sample to be analysed which contains magnetic particles, so that a magnetic field is subsequently applied in said reaction chamber, to retain the magnetic particles inside said chamber, the rest of the sample flowing out of the chamber, where a PCR reaction is subsequently produced controlling the temperature by means of heating associated to said chamber. Finally, the sample retained in the reaction chamber is optically detected.

The method can be applied, for example, to microbiological, clinical, food samples, etc.

The invention provides a portable and autonomous detection device which permits the specific identification of genetic markers by the real-time PCR technique, automatically, including in the chamber the concentration and preparation of the sample, the amplification and the optical detection. The miniaturized system increases the efficiency, simplicity of use and portability of the PCR in comparison with analysis on a laboratory scale. The micro-device makes it possible to quickly diagnose the presence of a determined sequence of oligonucleotides (DNA and RNA), by the technique of real-time PCR in a final volume less than 10 microlitres and in less than 30 minutes.

The temperatures necessary to carry out the real-time PCR are achieved by an original system of integrated heating close to the reaction chamber, which maintains the temperature homogeneously throughout the chip during the different cycles the PCR consists of. Said temperature is preferably in the range of ambient temperature and 95° C.

### DESCRIPTION OF THE DRAWINGS

To complement the description being made, and in order to help towards a better understanding of the characteristics of the invention, in accordance with a preferred example of practical embodiment thereof, a set of drawings is attached as an integral part of said description, wherein, with illustrative and non-limitative character, the following has been represented:

FIG. 1 shows an exploded view of the elements that form the encapsulation of the PCR device.

FIG. 2 figure (a) is a schematic and sectional representation of the PCR micro device encapsulation, and figure (b) is a representation similar to that of figure (a) but in a phase prior to the encapsulation.

FIG. 3 shows two perspective views of the PCR device, wherein it illustrates the possibility of placing and removing the magnets during the process. Figure (a) shows the upper magnet placed in the openings of the upper base, whilst figure (b) shows the magnets outside of the device.

FIG. 4 figure (a) is a schematic representation of a plan view of the sealed chamber of the PCR chip where the means of heating, the electric contacts and the rhomboidal contour of the chamber are observed. Figure (b) is a representation of the heating electrode and the temperature sensor.

FIG. 5 is a schematic and sectional representation of the sealed chamber.

FIG. 6 is a diagram of the 4-wire resistance sensor used to measure the temperature in the centre of the chamber.

FIG. 7 shows a diagram corresponding to a microfluidic circuit and the PCR micro-chamber.

FIG. 8 is a schematic representation of a plan view of one of the heaters in the form of elongated plate.

FIG. 9 represents a simulation (ANSYS) of the current distribution on the heating plate of FIG. 8. The irregular forms at the ends indicate the distribution of current on the surface.

FIG. 10 is a perspective representation of the injection process of a sample to be analysed in the PCR micro-device.

FIG. 11 is an upper plan view of the device, where the capsules of the upper and lower bases are observed which are used to connect the device fluidically and electrically.

FIG. 12 represents a diagram of the manufacturing process of the Ti/Pt microelectrodes on a Pyrex substrate.

FIG. 13 represents a diagram of the manufacturing process of the seed layer of SU-8-5 on the pyrex substrate with Ti/Pt electrodes.

FIG. 14 represents a diagram of the manufacturing process of the cavities of the PCR micro-chambers on the pyrex substrate with Ti/Pt electrodes and the seed/insulating layer of SU-8-5.

FIG. 15 represents a diagram of the manufacturing process of the covers of the PCR chambers on a Kapton film.

FIG. 16 represents a diagram of the process of adhering of the two substrates.

FIG. 17 shows a graph of the results of a concentrated sample, which is lysated and thermocycled inside a chip.

FIG. 18 shows the verification through running the sample taken from the chip in an electrophoresis gel.

FIG. 19 shows a perspective view of the PCR device.

FIG. 20 shows another preferred embodiment of the invention where the encapsulation is a portable box and the reaction chamber is disposed in a plastic support. The figures (a, b and c) are three perspective views of the box in the open position.

FIG. 21 shows an exploded view of the sheets that form the plastic support holding the PCR chip.

### PREFERRED EMBODIMENT OF THE INVENTION

The device object of the invention comprises a reaction chamber (1) connected to two micro-conduits. An inlet micro-conduit (2) whereby the sample to be analysed is introduced and an outlet micro-conduit (3), which allows the flow towards the exterior.

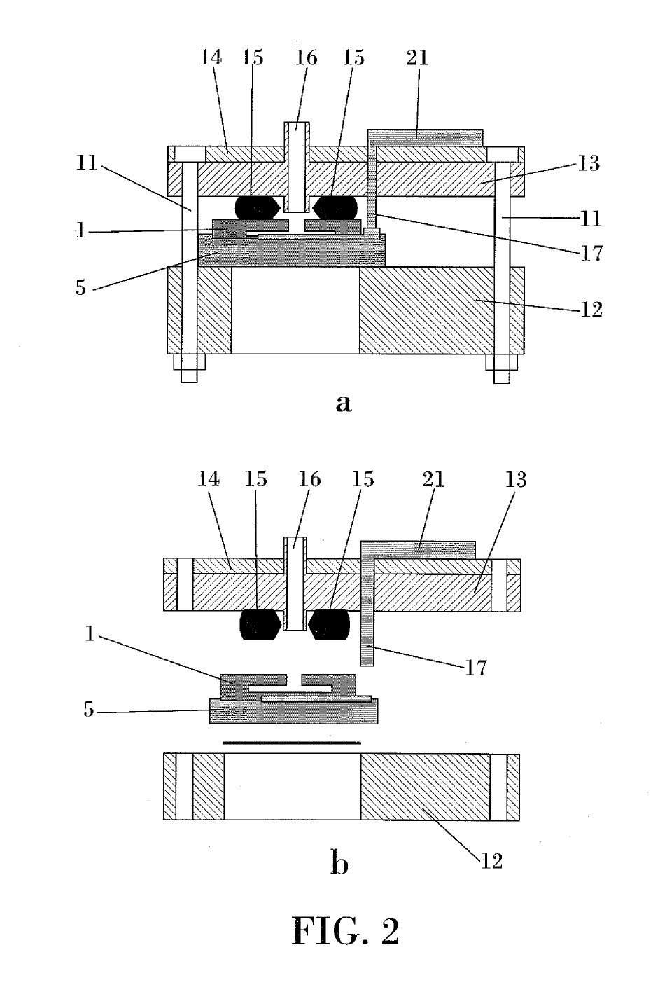

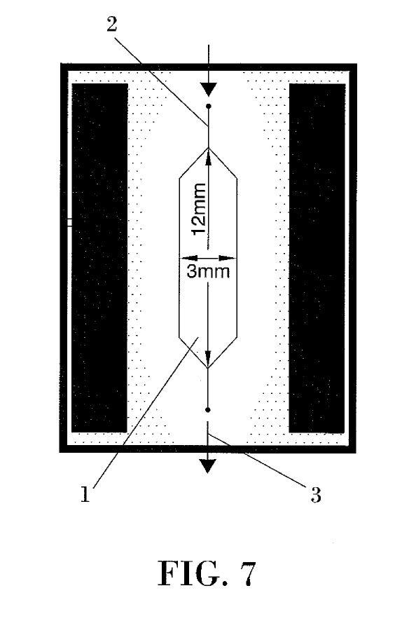

In a preferred embodiment of the invention, this chamber has dimensions of 12×3 mm as indicated in FIG. 7, and is elongated with a central portion with rectangular plan and triangular end portions in correspondence with the inlet and the outlet to facilitate the injection/extraction of the PCR mixture. The micro-conduits (2,3) connected to the chamber (1) have a length of approximately 2.5 mm and a width of 70 μm and end in a connection to the outside which makes it possible to introduce and evacuate the liquid within the fluidic circuit as observed in FIG. 2.

The device incorporates, in this preferred embodiment, integrated heating elements solidly joined to the reaction chamber (1) disposed so that they can heat said chamber uniformly and controlled by external means.

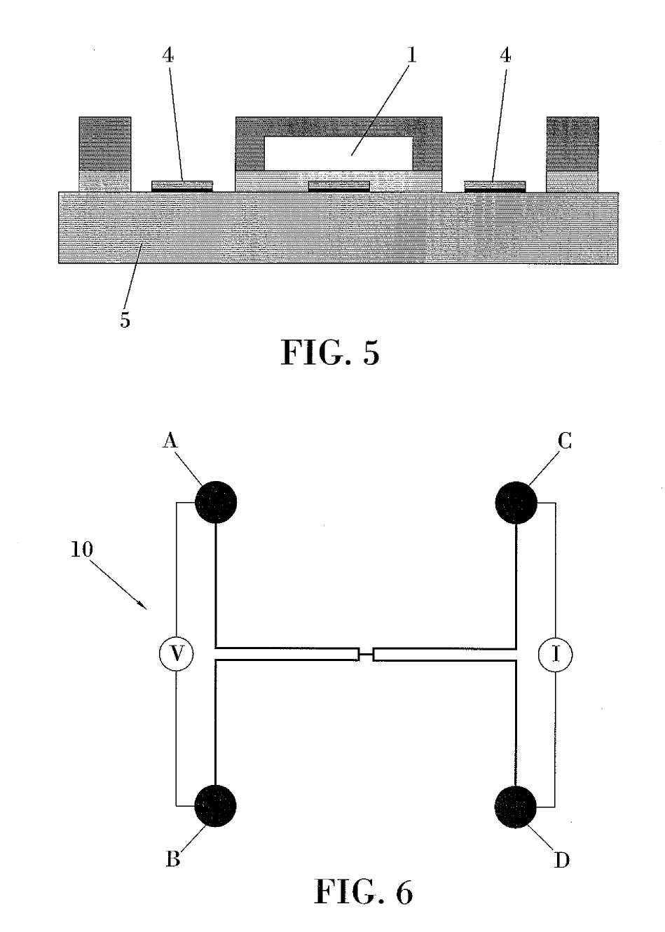

The microfluidic circuit formed by the reaction chamber and the two micro-conduits is formed preferably on a substrate of 4.5 μm of SU-8-5 which serves to electrically insulate the terminals (4) for the electrical supply of the means of heating from the liquid. The height of these chambers may vary depending on the sample volume which one wants to thermocycle (from 120 μm to 400 μm). This microfluidic circuit is produced from a photodefinable exoxy resin called SU-8-50 which is deposited on a Pyrex substrate (5), as shown in FIG. 5.

Alternatively, the substrate can be produced by another polymeric substrate (PMMA, SU-8, COC).

The means of heating comprise a plurality of conductive wires (6) connected between two terminals (4), and, preferably, they are produced by a sheet of titanium (Ti) superimposed on a sheet of platinum (Pt).

The wires (6) are parallel throughout their length, and they are arranged equidistant to one another.

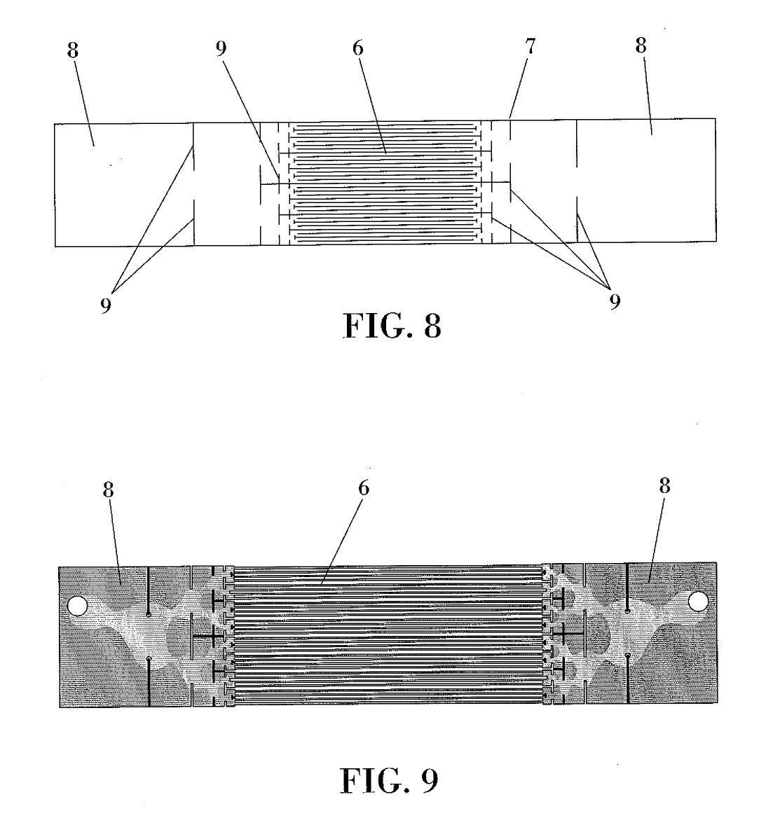

The heating wires (6) are formed in a heating plate (7) of conductive material and with elongated form, which has a connection surface (8) at each one it its ends, and wherein is connected respectively a first and a second connection terminal. Said conductive wires (6) are straight and are connected between said connection surfaces (8), as shown in FIG. 8.

Said connection surfaces (8) have transversal cuts (9) in the form of a straight line which define conductive paths of current to produce a uniform distribution of current in the conductive wires (6), as shown in the simulation of FIG. 9.

As observed in FIG. 8, said conductive paths are defined by at least two parallel groups of aligned cuts (9). These cuts (9) achieve that the distribution of the current is uniform irrespective of the position in which the flexible electrical points (17) are found and with which each terminal of the means of heating is supplied, as observed in FIG. 2(b).

FIG. 9 shows the simulations performed in ANSYS which demonstrate that the maximum variation of the current between two heaters of this type does not exceed 0.04 μA.

The design of the heating elements incorporates two compensation structures with the same purpose, but to resolve different problems:

Compensation of the geometry. The electric current always tries to go through the path of least electrical resistance. Due to the symmetrical geometry of the heater, all the resistances, which are located in a parallel configuration, have the same resistance. However, although the area of the external electric contact has been made very large in order so that there is no predominant direction, as it is made with the same material as the resistance, it has a certain electrical resistance. This means that there is a preferred path, the central (if the contact is centred). Due to the required level of uniformity of temperatures due to the application, that small lack of uniformity is not acceptable, for which reason balanced current structures have been designed and simulated between the different branches. These structures have the function of equalling the total path in all resistances so that there is not one more favourable than the others. These structures are T-shaped. The central area of the T has the aim of cutting a short path and deviating the current through the sides. 4 levels have been added, this number depending on the degree of uniformity required. As it goes up a level, the “T” is of greater size (25%), covering a greater surface. Each arm of the T is equivalent to the distance between this and the previous T.

Compensation of the contact. If the electric contact to the heater is not performed in centred form, there is a lack of uniformity and it favours a more comfortable path. To minimize this effect, lateral cuts are introduced which oblige the current to go towards the central zone of the heater. The opening should be a third of the opening which is seen in the last line of “T” (the closest to the electric contact). The separation between this opening and the closest line of the “T” should be so that the angle opened is 25°.

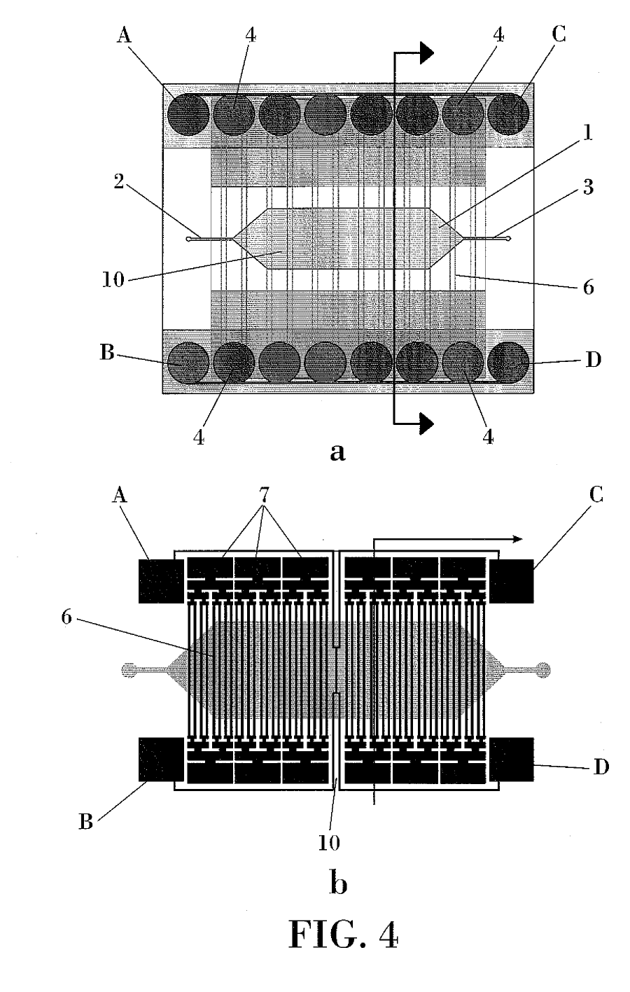

In a preferred embodiment, the PCR device has six heating plates (7) placed in parallel as shown in FIG. 4(b), and preferably transversally to the reaction chamber (1). Each heating plate (7) has 32 tracks of wires (6) of Pt of 20 μm width and 5 mm in length, separated 50 μm from one another, which end in two electric contacts (one on each side of the chip), through which they are supplied at 45 V by an external power source. It has conductive wires (6) under the whole area of the reaction chamber (1) as is observed in FIG. 4, so that all zones of the chamber are uniformly heated.

In addition to these six heaters, the PCR device contains a temperature sensor, in particular a resistance sensor (10) of four wires, placed in the centre of the reaction chamber (1) as shown in FIG. 4. In this way, from the 16 electric contacts that can be seen in FIG. 4, the contacts (A,B,C,D) are those belonging to the temperature sensor. This resistance sensor (10), shown in FIG. 6, is based on the principle that the electrical resistance of the platinum depends on the temperature and varies linearly with it. When the sensor is supplied with a voltage of 4.5 V through contacts (A) and (B) and the current is measured through contacts (C) and (D), it is possible to calculate the resistance and therefore the temperature in the centre of the PCR chamber.

The heating wires (6) are immersed in one of the walls that form the reaction chamber (1). The wires can go on the pyrex or on the SU-8. In a preferred configuration the wires go on the pyrex and coated with a layer of SU-8-5.

For the use of the device it is necessary to fill the chamber with a PCR mixture, and then close the inlet and outlet micro-conduits (2,3) of the chamber (1) by the use of an encapsulation with Silicon seals which close the inlet and outlet orifices.

As represented schematically in FIG. 2, the encapsulation is based on two capsules or bases pressed together with screws (11), which leaves the reaction chamber (1) in the middle.

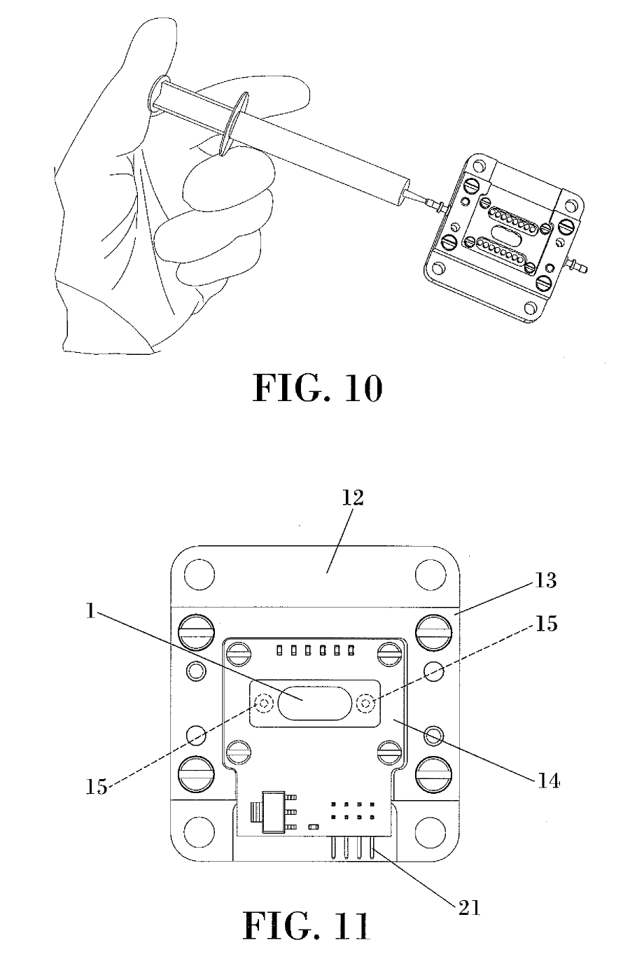

The lower capsule (12) acts as support of the reaction chamber (1) formed on the substrate (5), whilst the upper capsule (13) acts as support for a PCB (printed circuit board) (14) and contains an o-ring (15) for each inlet/outlet of the chamber (1), as well as two orifices (16) which place in contact the inlet/outlet of micro size of the device with connectors of greater size whereto can be joined a tube or syringe as shown in FIG. 10.

Furthermore, the upper capsule (13) places in contact through retractile electrical points (17) in its interior, the contacts of the PCB (14) with the electrodes of the PCR device, so that through electric contacts (21) existing in the PCB (14) it is possible to supply the means of heating by an external power source.

On aligning by hand all the pieces and tightening using screws, the (11), the PCR chip is fluidically and electrically capsulated without the need for adhesives, so that it is possible to easily replace and connect the PCR device, it being possible to easily use the same encapsulation for different chips. On the other hand, the lower capsule and the upper capsule respectively have an upper opening (18) and a lower opening (19), both the size of the chamber (1) to, on the one hand, place magnets (20) on the surface of the chip and, on the other hand, have visual access to the inside of the chamber (1) when the fluorescence is measured.

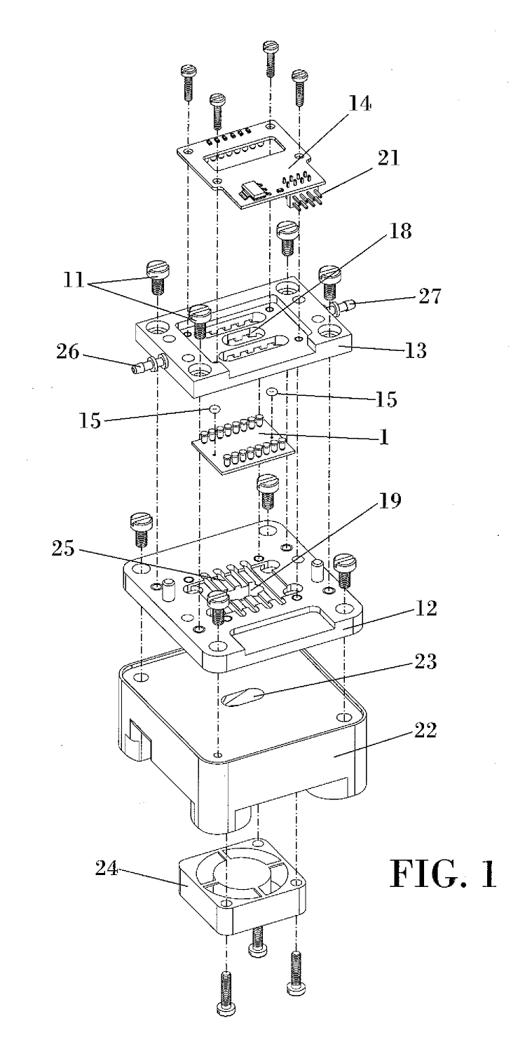

FIG. 1 shows the process to encapsulate the device, fluidically and electronically. In this figure, the upper capsule or base (13) and the lower capsule or base (12) can be seen, made in PMMA, with screws (11) and pins to facilitate the alignment. The PCB (14) is also observed with various electronic components to supply the means of heating and the electrical connector in the lower part.

An injection of fluids without leaks is achieved via o-rings (15), which passes through the upper capsule or base (13) to the reaction chamber (1) by internal conduits (28,29) of said capsule.

The device encapsulation is mounted on a support (22) which has a central opening (23), so that a fan (24) is coupled at its lower part to be able to more quickly cool the reaction chamber. It can be observed that both capsules have grooves (25) which favour the passage of air driven by the fan to facilitate the cooling by forced convention.

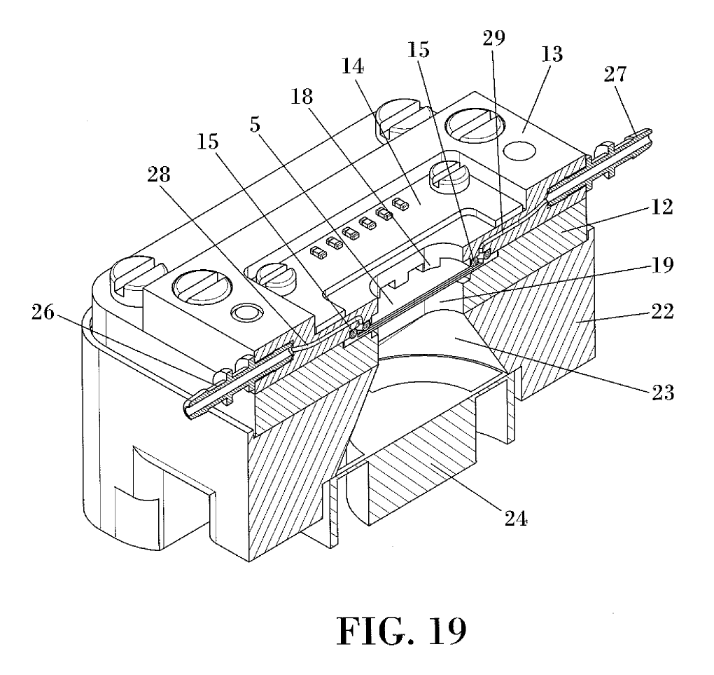

The upper capsule or base (13) has an inlet conduit (26) and an outlet conduit (27), which connect respectively with said inlet and outlet micro-conduits (2,3) of the reaction chamber (1), through the internal conduits (28,29) as shown in FIG. 19.

Once the device is encapsulated, and when performing the detection process, the sample is introduced in the reaction chamber (1), for example, by a syringe as represented in FIG. 10.

In the invention it has been provided that the encapsulation should permit the placement of the magnets (20) very close to the chip, i.e. to the reaction chamber (1), in addition to not hindering its cooling or the light beam. To do this, the openings (18) and (19) of the upper and lower capsules, make it possible to place the magnets (20) inside them so that they can be later removed.

For the preparation of the sample a universal concentration system is used which permits the capture and concentration of biological samples (microbiological, clinical, food, environmental, etc). This system can use superparamagnetic particles coated by specific antibodies or superparamagnetic particles that specifically attract nucleic acids. On placing the test sample in liquid state with the magnetic particles it achieves a preconcentration of the fraction which contains the specific sequence for the PCR reaction.

A volume of the sample which can be analysed (1-3 ml), is made to pass through the micro-chamber (1) whist applying a magnetic field thereto. In an example of embodiment the volume of the sample can be between 1-10 ml.

Once the sample has been introduced in the chamber through the inlet, it is displaced, by the movement of the syringe plunger, throughout the chamber to the outlet where it is eliminated to the outside of the encapsulation maintaining the magnetic field. The sample exits the chamber (1) through the micro-conduit (3) which connects with the outlet conduit (27) through the inner conduit (29). The gasket seal (15) avoids any leak in the passage of liquid between the micro-conduit (3) and the inner conduit (29).

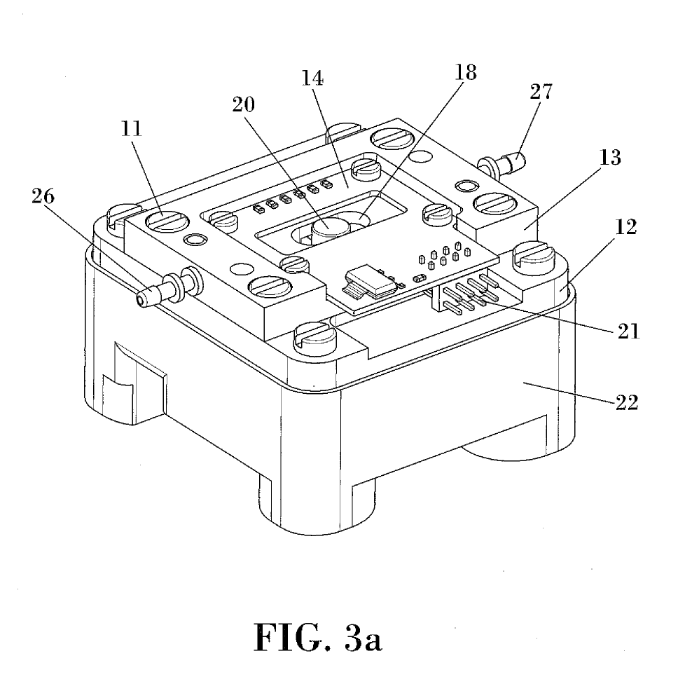

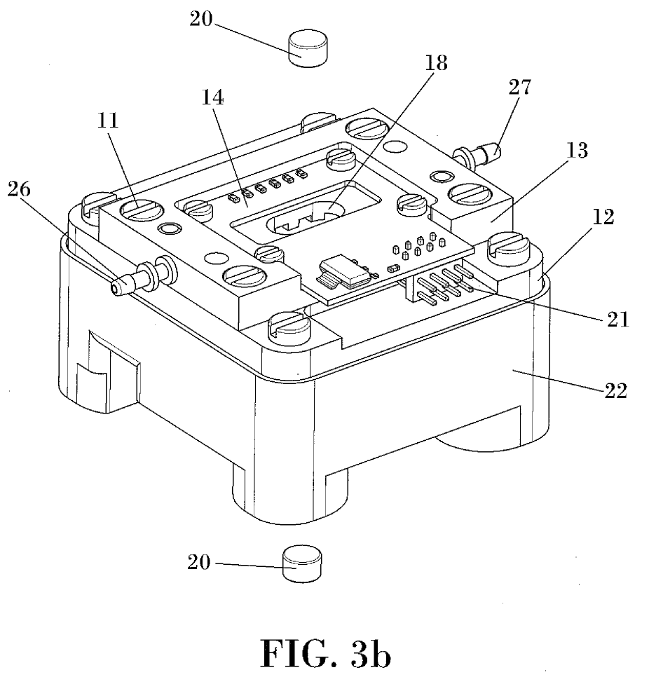

The magnetic field is applied on placing the two magnets (20) on the reaction chamber (1), one in the upper part and another in the lower part, as shown in FIG. 3. To do this, the encapsulation makes it possible to place the magnets very close to the micro-chamber. In this case, the upper magnet is in contact with the cover of the micro-chamber, which has an approximate thickness between 70 μm and 100 μm, which makes it possible to perform an extremely efficient magnetic capture due to the proximity of the magnet. The lower magnet is in contact with the pyrex substrate (5), which has a thickness between 750 μm and 750 μm.

In other preferred embodiments, it is possible to eliminate this substrate, using the process described in patent IS-2,263,400, which would permit a proximity less than 200 microns between the magnet and the reaction chamber.

In this way, as the sample is made to pass through the chamber (1), only the magnetic particles and the magnetic particles-target analyte complex, if any, are retained therein. Once the target is captured inside the chamber (1) of the chip and having ensured the total absence of fluid inside the chamber (1), the PCR mixture is then introduced in the same reaction chamber (1), the magnets (20) are removed to then proceed with the amplification reaction.

For the preparation of the sample the following universal systems can be used which permit the capture and concentration of genetic material: superparamagnetic particles (DYNAL©) which specifically trap nucleic acids and superparamagnetic particles coated by covalent bonding, by specific antibodies to a target analyte. On placing the test sample in contact with the magnetic particles, these specifically bond to their target if this is found in the sample, so that the magnetic particles-target analyte complex is formed. The sample, with this complex, is introduced through the inlet of the encapsulation and is made to pass through the reaction chamber (1) where, when necessary, the biomolecules (DNA and RNA) and the PCR reaction is performed and, at the same time, a magnetic field is applied which retains the magnetic particles inside the micro-chamber. In this way, after the passage of the solution, only the magnetic particle-target analyte complex, which includes the specific sequence, which serves as mould for the PCR reaction, is retained in the chamber.

Once the target analyte is captured inside the chamber of the chip, the PCR mixture is introduced therein. The chip, encapsulated and perfectly closed, is placed under an epifluorescent microscope or a CCD chamber of a photomultiplier with the respective optical filters which permit measuring the fluorescence.

When the amplification protocol is applied, in the case that for the concentration magnetic particles with antibody were used, the pre-activation time necessary for the polymerase enzyme is sufficient to provoke the lysis of the target analyte, contained in the chamber in the form of magnetic particle-antibody-analyte complex and leave accessible the nucleic acid (DNA and RNA) for its subsequent detection by amplification.

The amplification program contains the temperature cycles corresponding to pre-activation of the enzyme and amplification (denaturing, hybridization and extension), in a range between ambient temperature and 95° C.

The formation of the amplification product by real-time PCR is observed in the chip, through the transparent coating of SU-8, and it is possible to use specific molecular probes for the product amplified and labelled at end 5′ with fluorophore, for example Cy5, at end 3′ with BHQ-2. (Cy5 is a registered trademark of GE Healthcare Bio-Sciences, Little Chalfont, United Kingdom. BHQ-2 is registered trademark of Biosearch Technologies, Inc., Novato, CV).

The fluorescence is measured during the amplification reaction using voltage units. When the sample is positive, an exponential increase in the fluorescence is observed until reaching a maximum. The start of this increase in fluorescence occurs from a certain cycle of amplification, which depends on the initial quantity of nucleic acid. The complete amplification protocol lasts no longer than 30 minutes.

Through the openings (18) and (19) of the encapsulation, the PCR reaction chamber (1) remains in contact with the air, for three main reasons: (i) To be able to place the magnets in contact with the chip; (ii) So that the cooling is quicker and (iii) To be able to perform the optical detection.

The magnets (20) are placed one under the other above the chamber, by hand, so that they fit through the openings (18) and (19) of the capsule for which reason it is very easy to concentrate the sample and extract the nucleic acid and remove them subsequently to be able to amplify the nucleic acid and be able to perform the optical detection.

On the other hand, the external electronic apparatus for the heating of the means of heating consists of:
(i) a voltage source which supplies the heating wires (6)
(ii) a voltage source which supplies the fan (24)
(iii) a data collection system that measures the resistance of the sensor (10)
(iv) a software to control the temperature.

The system of heating works as follows: in first place, the sensor of the chip measures the resistance (and with it the temperature of the chamber) and according to the temperature needed at any time, it is decided whether to supply the heaters or the fan. If the temperature measured is less than needed at that time, the voltage source which supplies the heaters switches on and heats the chamber until reaching the desired temperature. But if, in contrast, the temperature measured by the sensor is greater than that needed at that time, the voltage source which supplies the fan switches on to cool the PCR chamber. All of this is controlled by software connected to the data collection system.

The data collection apparatus is based on a microscope and contains:
(v) a light source that consists of a 100 W mercury lamp
(vi) an excitation filter which filters all the wavelengths, except 640 mm (wavelength which excites fluorochrome Cy5)
(vii) a dichroic mirror that sends the light emitted by the sample towards the emission filter
(viii) an emission filter that filters all the wavelengths, except 670 mm (wavelength emitted by fluorochrome Cy5)
(ix) a photomultiplier or a CCD chamber which collects the light that passes through the emission filter

The viewing of the amplified nucleic acid is possible thanks to the accumulation for each amplification cycle of flurochrome Cy5, which is excited at 640 mm and emits at 670 mm.

The light emitted by the mercury lamp passes through the excitation filter. This only lets the light of 640 mm pass through, which reaches the sample. In consequence, the flurochrome is excited and emits a red light of 670 mm which is deviated towards the emission filter, thanks to the dichroic mirror. Finally, this emission light reaches the photomultiplier, which is connected to a data collection system.

As previously explained, the upper capsule or base (13) has an orifice (18) situated above the chamber (1), so that it permits this type of optical detection, since the cover of the micro-chamber is transparent. Furthermore, it is important to highlight that the SU-8, unlike other polymeric materials, has a very low autofluorescence at this wavelength, so that it makes it possible to detect the fluorescence signal of the sample labelled with Cy5.

The magnets (20) used for the preparation of the sample are Neodymium-Iron-Bro (NdFeB) and they have the shape of a disc, as observed in FIG. 3b, with a diameter of 10 mm and a height of 4 mm. The orientation of the magnetization is axial with a (B-H)ₘₐₓ of 30 MGOₑ.

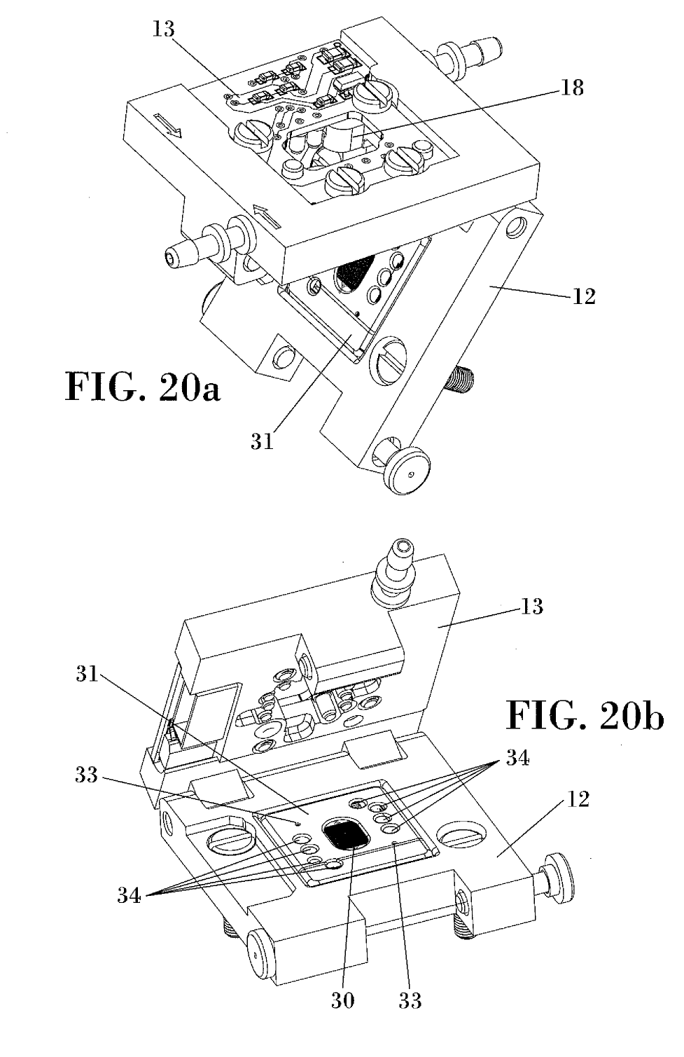

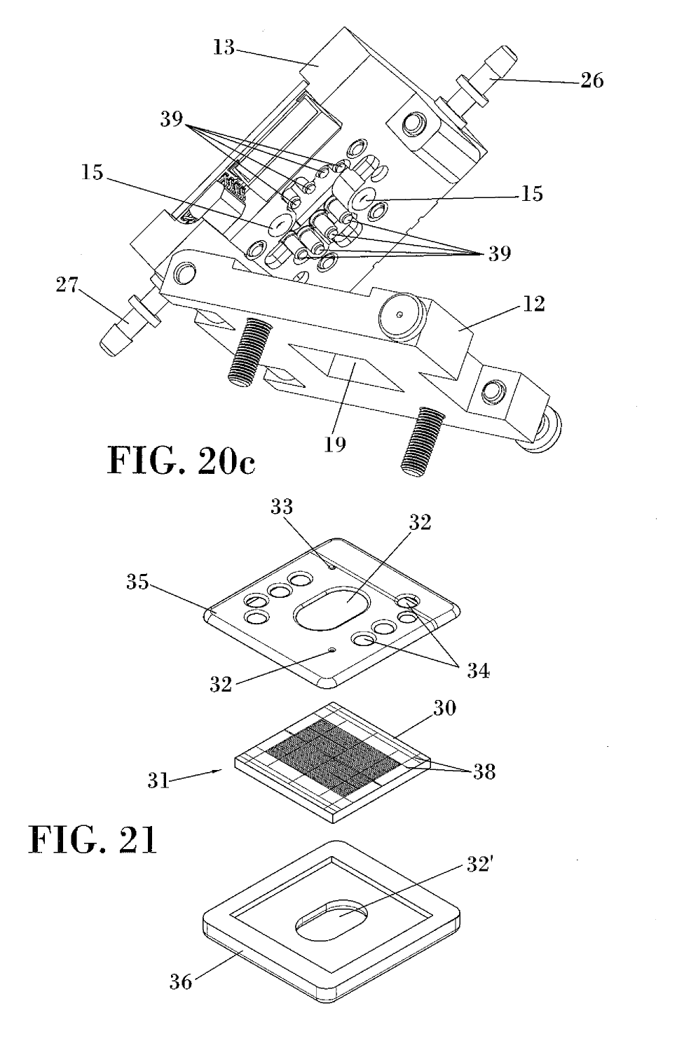

FIG. 20 has represented another preferred embodiment of the invention, wherein the upper base (13) and the lower base (12) of the encapsulation, are joined in hinged or articulated form at one of their sides, forming a portable device of small dimensions. The PCR (30) chip, which includes the reaction chamber (1), the micro-conduits (2,3) and the means of heating, is embedded in a plastic support (31) which has windows (32,32′) respectively on its upper and lower faces, which give access to the chip (30) as shown in FIG. 21.

In one of the surfaces of the plastic support (31) are arranged two orifices (33) connected inside the plastic support with the micro-conduits (2,3). The plastic support also has orifices (34) on one of its surfaces which give access to electric terminals (38) connected to the means of heating and the temperature sensor of the chip (30).

The plastic support (31) is formed by several sheets as observed in FIG. 21. In particular, it has an upper sheet (35) and a lower sheet (36), between which is arranged in a sandwich type structure, the chip (30).

Between the upper or lower bases (13) and (12) is defined a space suitable to receive the plastic support (31). Once introduced, the plastic support closes the encapsulation bases, so that the conduits (26,27) disposed on one of the bases, are connected to the orifices (33) of the plastic support (31). Similarly, electric contacts (39) are placed inside one of the bases to contact with the terminals (38) on closing the encapsulation.

For the placement of the magnets there is also openings (19) and (18), in the upper or lower bases (13) and (12).

A fan can be positioned in one of the bases, to drive air with the object of reducing the temperature of the reaction chamber when necessary.

### 1.—Manufacturing Process of the Device

In a preferred embodiment of the invention, the PCR devices are manufactured on pyrex substrates. However, it is possible to manufacture them on polymeric substrates such as, for example, PMMA as is described in patent IS-2,255,463 and without any substrate apart from the SU-8 in patent IS-2,263,400, so that its manufacturing cost is considerably reduced.

To manufacture the PCR devices on pyrex substrates it is necessary to carry out three fundamental steps: (i) Manufacturing of electrodes on pyrex substrates, (ii) Manufacturing of the seed layer of SU-8-5 and (iii) Manufacturing of sealed micro-chambers. Each one of these steps is explained in more detail in the following sections.

### 1.1. Manufacturing of Electrodes

We start with a pyrex substrate whereon is carried out a photolithography process with the positive photoresin S1818, using the appropriate mask. To do this, an adherence promoter is first deposited and then the resin at 4000 rpm during 30 seconds, the substrate is subjected to a thermal treatment at 90° C. during 20 minutes, it is exposed to UV light with a dose of 300 mJ/cm² and it is removed.

Then, 15 nm of titanium (3 minutes at 100 W) and 140 nm of platinum (6 minutes at 190 W) are deposited using the cathode spray method throughout the pyrex substrate. Finally, the substrate is introduced in an acetone ultrasound bath and on dissolving the photoresin S1818, the metal remains only where there was no resin, thus producing the microelectrodes.

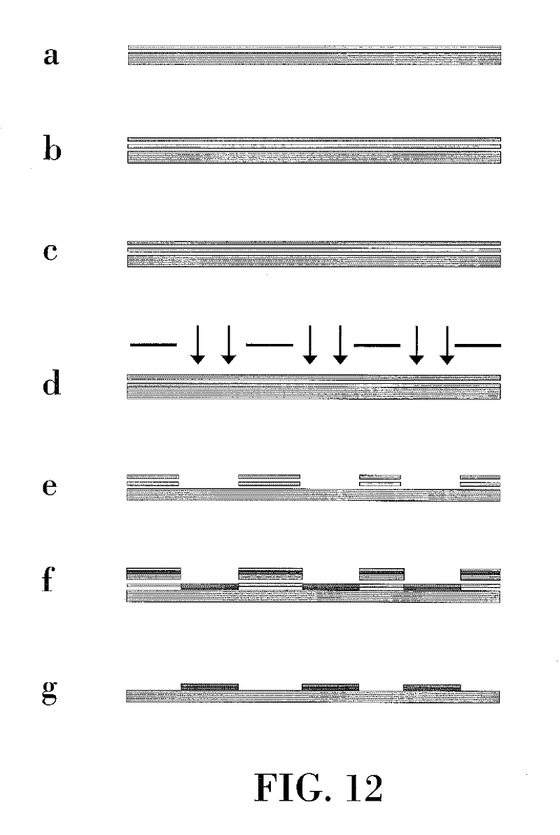

This part of the manufacturing is shown in FIG. 12, and is composed of the following phases:
a.—deposit of the adherence promoter of S1818 by centrifugation.
b.—deposit of the S1818 by centrifugation
c.—polymerization of the S1818 (20 minutes at 90° C.)
d.—exposure of 300 mJ of UV light to degrade the S1818
e.—removal of the degraded S1818
f.—deposit of 15 nm of Ti and 140 nm of Pt by cathode spray
g.—dissolution of the S1818 in acetone.

### 1.2. Manufacturing of Sealed Chambers

We start with two different substrates: the lower substrate is the same pyrex substrate where the electrodes have previously been manufactured and the upper substrate, which is a Kapton film adhered to a pyrex substrate.

### 1.2.1. Manufacturing of the Lower Substrate

We start from the pyrex substrate with the Ti/Pt electrodes produced after the process described in section 1.1. It is cleaned carefully in ultrasound baths of acetone, methanol and water respectively, to ensure that all the photoresin S1818 has been cleaned. Next, the seed layer of SU-8-5 is manufactured on this substrate with two objectives: (i) to electrically insulate the electrodes and (ii) to improve adherence between the pyrex substrate and the chambers manufactured in SU-8-50.

SU-8-5 and the SU-8-50 are chemically similar, the only difference existing between these two commercial products is the viscosity, which depends on the quantity of solvent they carry. The viscosity of the SU-8-5 (approximately 290 cSt) is much less than that of the SU-8-50 (approximately 2250 cSt). Therefore, the thickness of the layer of SU-8-5 is much less after being deposited by centrifugation on the pyrex substrate. The adherence of this fine layer is better than the adherence of a thicker layer of the same material. Furthermore, it is necessary to bear in mind the degree of polymerization. The greater the degree, the better the adherence between the substrate and this layer of polymer. Therefore, when manufacturing the seed layer, a fine layer of SU-8-5 (4.5 μm thickness) is deposited and it considerably polymerizes.

For this, 2 ml of photoresin are poured on the substrate and it is rotated at 3000 rpm during 30 seconds. The resin is spread throughout the substrate and a continuous layer of SU-8-5 of 4.5 μm thickness is produced. Next, the substrate is subjected to a thermal treatment of 95° C. during 5 minutes to evaporate the whole solvent. In this way only the prepolymer is read to be polymerized. To do this, the photolithography step is carried out irradiating the SU-8 with the UV light using the appropriate mask, with a dose of 160 mJ/cm². In this way, free radicals are created only in the parts coinciding with the clear areas of the mask. It is here where the polymerization starts and is propagated during the following thermal treatment, on maintaining the layer of SU-8-5 at 95° C. during 5 minutes.

Finally, the substrate is immersed in a PGMEA bath with stirring during 2 minutes and it is rinsed with IPA. In this last step, the photoresin which has not been polymerized is dissolved, the seed layer of SU-8-5 remaining on the pyrex substrate. However, the adherence is improved as the degree of polymerization of the photoresin is increased. Therefore, the substrate is subjected to a last thermal treatment (30 minutes at 170° C.) wherein this degree of polymerization considerably increases.

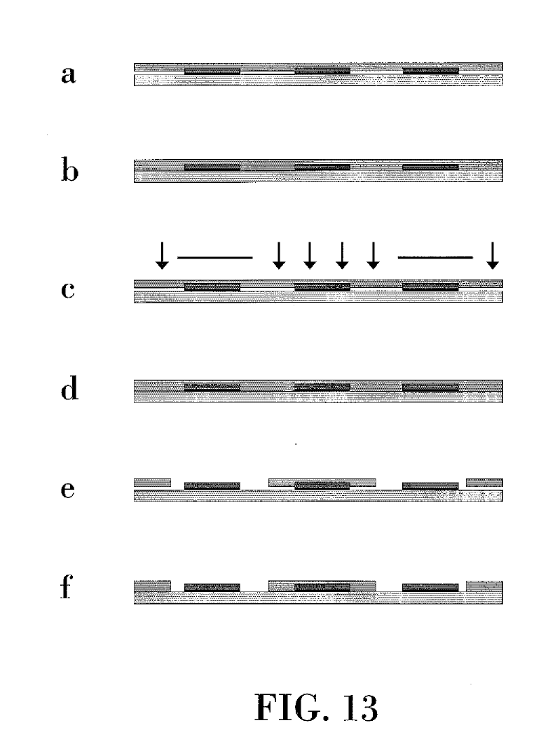

This part of the manufacturing is shown in FIG. 13, and is composed of the following phases:
a.—deposit of the SU-8-5 by centrifugation
b.—evaporation of the solvent at 95° C. during 5 minutes
c.—exposure of 160 mJ of UV light to start the polymerization
d.—propagation of the polymerization at 95° C. during 5 min.
e.—developing of the non-polymerized SU-8-5 in PGMA
f.—high polymerization at 170° C. during 30 minutes

Once this seed layer is polymerized, the cavities of the PCR chambers can be manufactured with their microchannels in it, by another photolithography process. But this time a thicker layer of SU-8-50 is used, which can vary between 20 and 200 μm of thickness, according to the height of chamber desired. Although some process parameters may change, the procedure to follow is similar. In first place 2 ml of resin are deposited and the substrate is rotated during a few seconds to produce a uniform layer. Then, the solvent is evaporated with thermal treatment at 90° C. Then the resin polymerizes by exposure to UV light and a thermal treatment at 90° C. Finally, the non-polymerized resin is developed to produce the desired structures. In this case, the degree of polymerization is relatively low so that it can continue polymerizing afterwards during the adherence process, in contact with another layer of SU-8.

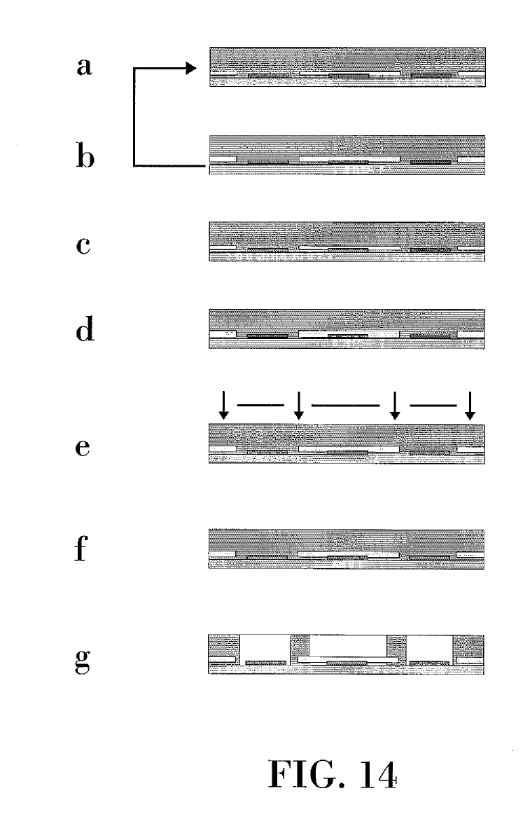

This part of the manufacturing is shown in FIG. 14, and is composed of the following phases:
a.—deposit of SU-8-50 by centrifugation (20, 37 or 80 μm in height)
b.—evaporation of the solvent at 90° C. during 8, 15 or 30 minutes depending on the height
c.—deposit of 20 μm of SU-8-50 by centrifugation
d.—evaporation of the solvent at 90° C. during 8 minutes
e.—exposure of 190 mJ of UV light to start the polymerization
f.—propagation of the polymerization at 90° C. during 4 minutes
g.—developing of the non-polymerized SU-8-50 in PGMEA

As has been explained in the previous paragraph, it is possible to produce thicknesses of SU-8 between 20 and 200 μm by the combination of different layers of 20, 37 and 80 μm in height. To do this, the deposit of layers of these three different heights has been optimized so that layers are produced with very good uniformity in the thickness, which is a critical parameter for a good subsequent adherence.

In the case of 20 μm, 2 ml of resin are deposited and the substrate is rotated at 6000 rpm during 60 seconds. Then the solvent is evaporated, subjecting the substrate to thermal treatment of 90° C. during 8 minutes.

In the case of 37 μm, 2 ml of resin are deposited and the substrate is rotated at 3000 rpm during 60 seconds. Then the solvent is evaporated, subjecting the substrate to a thermal treatment of 90° C. during 15 minutes.

Finally, in the case of 80 μm, 2 ml of resin are deposited and the substrate is rotated at 1500 rpm during 60 seconds. Next, the solvent is evaporated, subjecting the substrate to a thermal treatment of 90° C. during 3 minutes.

Different combinations may be made between these three layers to produce the desired chamber height. For example for a chamber of 100 μm in height, 80 μm are deposited, the solvent is evaporated at 90° C. during 30 min and 20 μm are again deposited, evaporating the solvent at 90° C. during 8 minutes.

However, it is important that the last layer deposited on the substrate is always 20 μm in height, since the subsequent adherence process is optimized for these layers of SU-8.

### 1.2.2. Manufacturing of the Upper Substrate

We start from a pyrex substrate wherein is adhered a 125 μm kapton film. These films are very flexible and it is impossible to perform a correct photolithography on them. It is necessary to previously adhere them to a rigid pyrex substrate. To do this, 4 ml of the S1818 resin are deposited on the pyrex and it is rotated at 3000 rpm during 30 seconds. Next, it is placed in contact with the kapton film and it is introduced in the vacuum Substrate Bonder (0.1 Pa). It is heated to 90° C. during 20 minutes and the film is reversibly adhered to the pyrex substrate. In this way, the kapton substrate is produced, which is sufficiently rigid to carry out the photolithography of SU-8

The photolithography on the kapton is carried out exactly the same as the photolithography of the cavities, but with the suitable mask.

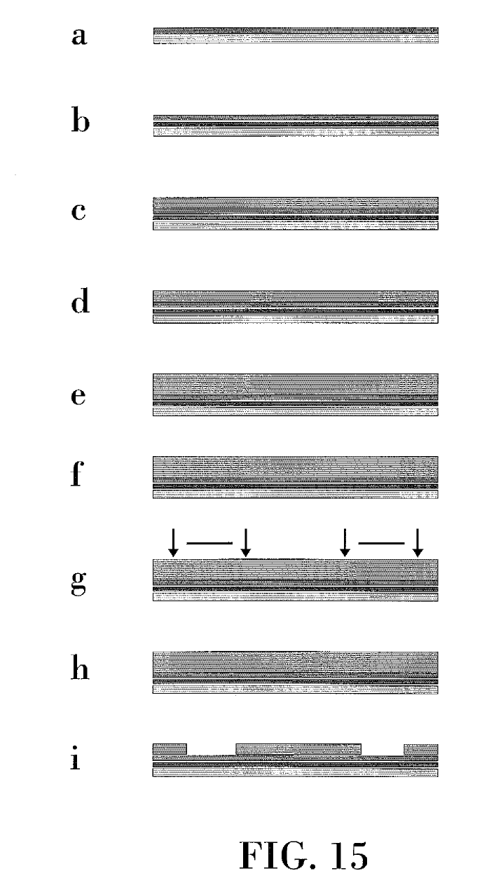

This part of the manufacturing is shown in FIG. 15, and is composed of the following phases:
a.—deposit of S1818 by centrifugation
b.—adhering of Kapton at 0.1 Pa and 90° C. during 20 min
c.—deposit of 80 μm of SU-8-50 by centrifugation
d.—evaporation of the solvent at 90° C. during 30 minutes
e.—deposit of 20 μm of SU-8-50 by centrifugation
f.—evaporation of the solvent at 90° C. during 8 minutes
g.—exposure of 140 mJ of UV light to start the polymerization
h.—propagation of the polymerization at 90° C. during 4 minutes
i.—developing of the non-polymerized SU-8-50 in PGMEA

In this case, a layer of SU-8-50 of 100 μm thickness is manufactured so that the cover of the PCR chamber is sufficiently rigid to support the pressure generated during the thermocycling. To do this, as has been explained in section 1.2.1, a layer of 80 μm is first deposited, its solvent is evaporated at 90° C. during 30 minutes. Next, a layer of SU-8-50 is again deposited at 20 μm and its solvent is evaporated at 90° C. during 8 minutes. After subjecting the pyrex-kapton substrate to 140 mJ of UV light with the appropriate mask and finally the layer is polymerized at 90° C. during 4 minutes.

All the thermal treatments carried out in these photolithography processes are carried out in ramps, since the sharp temperature changes make cracks appear in the SU-8 due to inner stress. Furthermore, these photolithography processes have been optimized by Taguchi techniques to produce uniform layers of SU-8 and with good adhesive properties. To do this, it is possible to adhere these layers to one another as explained in section 1.2.3.

### 1.2.3. Adherence of Structured Layers of SU-8

We start from the two photolitographied substrates in sections 1.2.1 and 1.2.2. As there are cavities in the lower substrate and covers in the upper substrate, after the adherence sealed PCR chambers are achieved. To do this, it is necessary to align the two structured layers before adhering them The kapton film used in this work is 125 μm thickness and it permits carrying out this alignment. The thicker this film is, the less transparent, and it is, for this reason that the 125 μm films have been chosen.

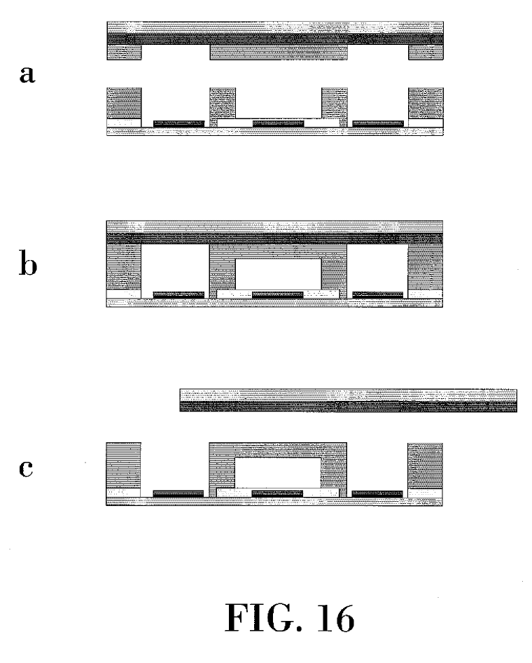

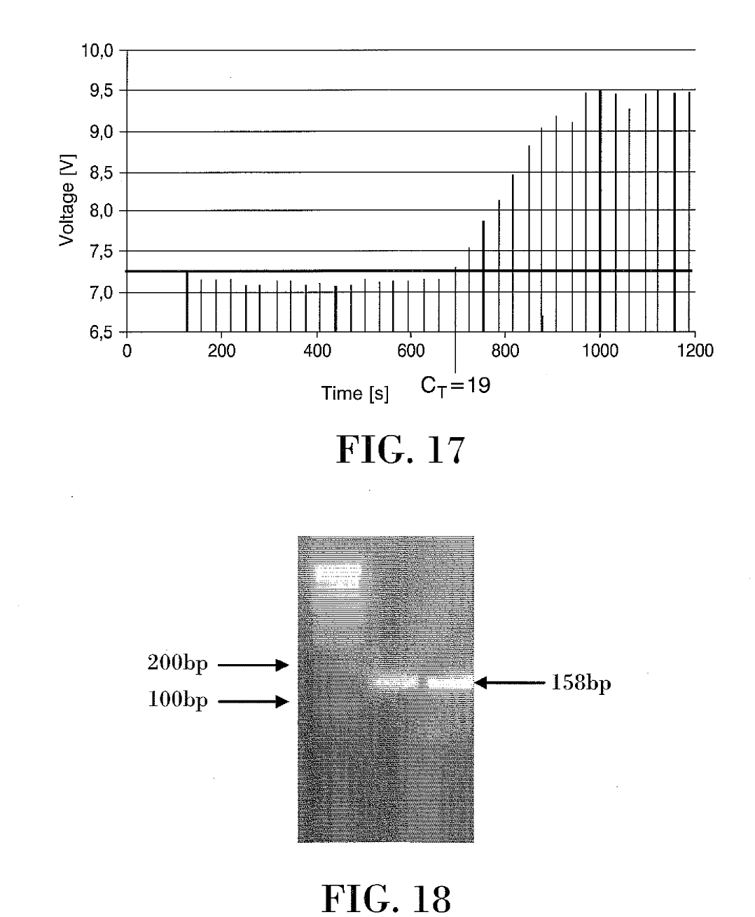

FIG. 16 shows a diagram of this manufacturing process, which is composed of the following operational areas:
a.—alignment of the two substrates
b.—adherence of the two substrates at 300 KPa and 100° C.
c.—release of the pyrex-kapton

As is explained in the diagram of FIG. 16, after the alignment the two substrates are introduced in the vacuum chamber of the substrate bonder at 0.1 Pa, and after placing them in contact, a force of 300 KPa is applied whilst the temperature is raised to 100° C. during 20 minutes. The two layers of SU-8 are irreversibly adhered.

Adherence between the kapton film and the SU-8 is very poor. Due to this, it is possible to release to upper substrate after the adherence process. To do this, the two substrates are introduced adhered in an IPA ultrasound bath during 10 minutes and the pyrex substrate is removed with the aid of a knife.

### 1.2.4. Cutting

After this release of the kapton, the two layers of SU-8 are produced adhered together on the pyrex substrate forming the sealed PCR chambers, with integrated platinum electrodes. In other words, a pyrex substrate is achieved which contains 16 PCR devices. Therefore, cutting this substrate in the cutter gives rise to 16 devices.

1-44. (canceled)

45. Device for the specific detection of genetic material by real-time polymerase chain reaction, which comprises a reaction chamber suitable for producing said polymerase chain reaction connected to an inlet micro-conduit and an outlet micro-conduit, respectively for the inlet and outlet of a sample to be analysed of said chamber, characterized in that it comprises:
a substrate wherein is formed said reaction chamber,
an upper base and a lower base, so that said substrate is retained between said upper and lower bases,
heating means arranged to heat said chamber and configured to produce the amplification of the genetic material in said reaction chamber,
a first opening in the upper base which gives access to an upper part of the chamber, and a second opening in the lower base which gives access to the lower part of the chamber,
a pair of magnets for producing the concentration of the genetic material in said reaction chamber, said magnets being adapted in size and shape to be housed with removable character respectively in said first and second openings,
wherein said first and second openings provide visual access to the inside of said reaction chamber to allow the fluorescent detection of the genetic material inside said reaction chamber,
a temperature sensor arranged to measure the temperature in said reaction chamber,
electric contacts situated in at least one of the upper or lower bases, electrically connected to said heating means and temperature sensor.

46. Device according to claim 45, characterized in that said substrate is retained with dismountable character between said upper and lower bases.

47. Device according to claim 45, characterized in that the means of heating are integrated in the substrate wherein the reaction chamber is formed and wherein said means of heating are arranged in the lower base.

48. Device according to claim 45, characterized in that the temperature sensor is integrated in the substrate wherein the reaction chamber is formed and wherein the temperature sensor is arranged in one of said bases.

49. Device according to claim 45, characterized in that at least one of the walls that form the reaction chamber is transparent.

50. Device according to claim 45, characterized in that one of the bases has an inlet conduit and an outlet conduit, that are respectively connected to said inlet and outlet micro-conduits of the reaction chamber.

51. Device according to claim 45, characterized in that the substrate wherein the reaction chamber is formed, the inlet and outlet micro-conduits, and the means of heating, is placed between plastic sheets forming a removable plastic support, and in that said plastic support has a window through which the reaction chamber is accessible, and wherein said plastic support has two perforations in one of its surfaces connected to the inlet and outlet micro-conduits of the reaction chamber, and wherein the plastic support has electric terminals accessible from one of its surfaces, which are connected to the means of heating immersed in said substrate.

52. Device according to claim 45, characterized in that said means of heating comprise a plurality of conductive wires connected between two terminals, at least one part of said wires being disposed substantially parallel to one another, and wherein the means of heating comprises at least one elongated conduction plate, which has connection surfaces on each one of its ends, and wherein is disposed respectively a first and a second connection terminal, and in that said conductive wires are straight and are connected between said connection surfaces, and wherein t each connection terminal is connected to the conductive wires through conductive paths defined in said connection surfaces, to produce a uniform current distribution in said conductive wires, and wherein said connection surfaces have transversal cuts in the form of a straight line which define said conductive paths, and wherein the device has at least two parallel groups of aligned cuts.

53. Device according to claim 52, characterized in that the conductive wires are arranged underneath the whole chamber surface.

54. Device according to claim 52, characterized in that one of the bases has electric terminals, which are in contact with said connection surfaces of the means of heating.

55. Method for the detection of genetic material by real-time polymerase chain reaction which comprises:
introducing a sample in a reaction chamber to be analysed and magnetic particles,
applying a magnetic field to said reaction chamber to retain the magnetic particles within said chamber,
extracting the rest of the sample outside the chamber,
producing a PCR reaction in the chamber controlling the temperature of the chamber by means of heating arranged to heat said chamber,
removing the application of magnetic field,
subjecting the sample in said chamber to an optical detection.

56. Method according to claim 55, characterized in that prior to the phase of producing a PCR reaction, a lysis is made of the cells by heating, and wherein prior to the phase of producing a PCR reaction, a chemical lysis is performed of the cells.

57. Method according to claim 55, characterized in that the optical detection in the chamber is performed by an epifluorescent microscope or equivalent system.

58. Method according to claim 55, characterized in that the control of temperature includes a first temperature cycle suitable to produce the pre-activation of the polymerase enzyme and to produce the amplification of the sample, if necessary.

59. Method according to claim 55, characterized in that the method is implemented in the device of claim 1.

60. Method according to claim 56, characterized in that the first temperature cycle of the PCR reaction produces the cell lysis.

61. Method according to claim 58, characterized in that the first temperature cycle of the PCR reaction produces the cell lysis.
